(19) United States
(12) Patent Application Publication
(10) Pub. No.: US 2008/0221670 A1
(43) Pub. Date: Sep. 11, 2008

(54) RADIOPAQUE POLYMERIC STENT

(76) Inventors: Claude Clerc, Marlborough, MA (US); F. Anthony Headley, Plymouth, MN (US); Forrest Whitcher, Allston, MA (US); John Damarati, Marlborough, MA (US)

Correspondence Address: HOFFMANN & BARON, LLP, 6900 JERICHO TURNPIKE, SYOSSET, NY, 11791, US

(21) Appl. No.: 12/037,342

(22) Filed: Feb. 26, 2008

Related U.S. Application Data
(60) Provisional application No. 60/905,460, filed on Mar. 7, 2007.

(51) Int. Cl. A61F 2/82

(52) U.S. Cl. 623/1.34; 623/1.53

(57) ABSTRACT

The invention relates to an implantable radiopaque stent adapted to be disposed in a body lumen. In one aspect of the invention, at least one radiopaque filament is arranged for permanent attachment to a hollow tubular structure. The filament is desirably arranged in a linear direction traverse to a longitudinal length of the structure, the structure having a tubular wall that defines an inner surface and an outer surface and opposing first open end and second open end. The radiopaque filament improves external imaging of the tubular structure on fluoroscope or x-ray imaging equipment.

### CROSS-REFERENCE TO RELATED APPLICATIONS

This application claims the benefit of U.S. Provisional Application No. 60/905,460 filed Mar. 7, 2007, the contents all of which are incorporated herein by reference.

### FIELD OF THE INVENTION

The present invention generally relates to an implantable stent, and more particularly, to radiopaque polymeric stents and methods for making the same.

### BACKGROUND OF THE INVENTION

Implantable stents are devices that are placed in a body structure, such as a blood vessel or body cavity, to provide support and to maintain the structure open. Generally, implantable stents made from metallic or polymeric wires or strands comprise a flexible tubular body composed of one or more rigid but flexible filament elements. Wire or filament stents have been formed into braids, weaves or knits using techniques suitable for such construction. In some stents, the filaments extend in helix configuration with a center line of the tubular body about a common axis. In braided constructions, the filaments can be interlaced to form a tubular body having a symmetrical arrangement of filaments, e.g. where the number of filaments in each direction of a braid is divisible by two. Generally, the greater the diameter of the tubular body, the more filaments are used to impart stability to the body.

Generally, the proper deployment of the stent in a body cavity, such as in a blood vessel, the esophagus or other body cavity, requires a medical practitioner to follow movement of the stent through the body to the precise position at which the stent is to be deployed. To that end, radiopaque stents have been developed that allow the medical practitioner to track the position of the stent during movement through the body using fluoroscope and/or x-ray devices.

The opacity of a stent image tends to vary with the material and type of process used to create the stent. For example, radiopacity may be limited by the location of radiopaque materials in or on the stent. Furthermore, introducing radiopaque materials into stent filaments can produce undesirable mechanical alterations to filament mechanical properties. As such, a minimal amount of radiopaque material is typically used in creating radiopaque stents to prevent undesired alteration of the physical properties of the stent.

Creating a stent with a minimal amount of radiopaque material, however, reduces the practioner's ability to track the position of the stent during movement through the body. As such, there exists a need for an improved radiopaque stent that has greater radiopacity, yet maintains its overall functionality during and after various medical procedures.

### SUMMARY OF THE INVENTION

The invention relates to an implantable radiopaque stent adapted to be disposed in a body lumen. In one aspect of the invention, at least one radiopaque filament is arranged for permanent attachment to a hollow tubular structure. The phrase arranged for permanent attachment” means that one or more radiopaque filaments are incorporated into the stent as a part of or all of the stent wall; for example, interweaving or braiding the filaments into a stent wall or interweaving or braiding the one or more radiopaque filaments with other filaments to form the stent wall; or attaching or joining the one or more radiopaque filaments to the stent by various means, such as by adheringly bonding it, or by looping it through the stent structure, or by mechanically fastening it to the stent structure. In some embodiments the radiopaque filament(s) is(are) present along substantially the entire length of the stent. In other embodiments the one or more radiopaque filaments are present along only one or more portions of the stent. In still other embodiments, the one or more filaments may be selectively positioned along one or more portions of the stent. In some embodiments the one or more radiopaque filaments are substantially, if not entirely, radiopaque along their length. In some embodiments, the one or more radiopaque filaments are radiopaque at the selective portions along their length.

The terms “wire” and “filament” as used herein includes polymeric and metallic wires and filaments, as well as composites made of either or both classes of materials.

In one embodiment, the filament is arranged in a linear direction traverse to a longitudinal length of the structure, the structure having a tubular wall that defines an inner surface and an outer surface and opposing first open end and second open end. The radiopaque filament improves external imaging of the tubular structure on fluoroscope or x-ray imaging equipment.

The stent of this aspect of the invention desirably may have a plurality of filaments arranged in a helix configuration about a centerline of the tubular structure with a common axis.

The stent of this aspect of the invention desirably may have the plurality of radiopaque filaments prepared by compounding a radiopaque powder with a polymeric material. Desirably, the radiopaque powder can be a metal, alloy, or ceramic, typically selected from the group consisting of gold, platinum, tungsten, platinum-tungsten, palladium, iridium, platinum-iridium, rhodium, tantalum or combinations thereof or barium sulfate, bismuth subcarbonate, bismuth oxychloride, bismuth trioxide. The radiopaque material may be encapsulated in another material and then incorporated into the filaments. Encapsulating the radiopaque material into another material may advantageously allow the radiopaque filaments to be formed easily and/or be less toxic.

Preferably, the polymeric material may be selected from polyester, polypropylene, polyethylene, polyurethane, polynaphthalene, polytetrafluoroethylene, expanded polytetrafluoroethylene, silicone, and combinations thereof.

The stent of this aspect of the invention desirably may have bioabsorbable and/or biodegradable material included in the radiopaque filament. The bioabsorbable and/or biodegradable materials may include poly-L-lactide, poly-D-lactide, polyglycolide, polydioxanone, polycaprolactone, and polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

The stent of this aspect of the present invention desirably may have filaments that terminate at the second end, wherein the filaments at the first end are arranged in a series of closed loops with each loop having an apex defined by a bend in one of the filaments and having an opposed base defined by crossing of adjacent filaments, and further wherein the apex of adjacent closed loops are longitudinally offset from one and the other.

The stent of this aspect of the present invention desirably may have filaments that are not arranged with closed loops and terminate at each of the first and second stent ends.

The stent of this aspect of the present invention desirably may have filaments that are arranged in any known manner in the art including weaving, knitting, braiding, twisting, tying, laser or electron beam etched, mechanically etched, molded, injection molded, layer deposition, dipped and other techniques.

The stent of this aspect of the present invention may also be partially or fully coated with a polymeric material. The stent may further include a hollow tubular graft disposed partially or fully over the interior or the exterior surface. Desirably, the graft is a polymeric material. The polymeric material may be selected from polyester, polypropylene, polyethylene, polyurethane, polynaphthalene, polytetrafluoroethylene, expanded polytetrafluoroethylene, silicone, and combinations thereof.

The stents of the invention may optionally include a polymeric coating which contains radiopaque particles. For example, a polymeric coating, such as a silicone, may include radiopaque particles dispersed therein. Once coated onto the stent, the coating serves its purpose as a coating as well as a radiopaque marker. The polymeric coating may serve to fill the spaces or openings in the stent, and the entire device serve as a coated stent or stent-graft.

The stents of the invention may optionally include a polymeric covering that contains radiopaque particles. For example, the polymeric covering may cover the entire stent and be formed by dipping the stent in the polymeric material.

In another aspect of the invention, a plurality of elongate radiopaque filaments are braided together to form a hollow tubular structure having a tubular wall that defines an inner surface and an outer surface and opposing first open end and second open end. The tubular structure optionally includes a polymeric cover that may include radiopaque particles, wherein the radiopaque particles and the radiopaque filaments improve external imaging of the tubular structure on imaging equipment, such as fluoroscopic or x-ray equipment.

In one aspect of the invention the radiopaque filaments are made from a metallic or polymeric core having a polymeric radiopaque coating over the wire core. For example, the wire may be spray coated or dipped in the coating and incorporated into the stent structure. In another embodiment, the filaments are polymeric and have the radiopaque material incorporated within the polymer. For example, the polymeric composition may include a radiopaque material, with radiopaque filaments being formed from the composition by, for example, extrusion.

The stent of this aspect of the invention desirably may have the radiopaque filaments prepared by compounding a radiopaque powder with a polymeric material. Desirably, the radiopaque powder is a radiopaque material selected from gold, barium sulfate, ferritic particles, platinum, platinum-tungsten, palladium, platinum-iridium, rhodium, tantalum or combinations thereof, and the polymeric material is selected from the group consisting of polyester, polypropylene, polyethylene, polyurethane, polynaphthalene, polytetrafluoroethylene, expanded polytetrafluoroethylene, silicone, polyacrylate copolymers, and combinations thereof.

The stent of this aspect of the invention desirably may have bioabsorbable material included in the radiopaque filament. The bioabsorbable material may include poly-L-lactide, poly-D-lactide, polyglycolide, polydioxanone, polycaprolactone, and polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

The stent of this aspect of the present invention desirably may have filaments that terminate at the second end, wherein the filaments at the first end are arranged in a series of closed loops with each loop having an apex defined by a bend in one of the filaments and having an opposed base defined by crossing of adjacent filaments, and further wherein the apex of adjacent closed loops are longitudinally offset from one and the other.

In another aspect of the present invention, a method for making a radiopaque stent is provided. The method includes the steps of (i) providing at least one radiopaque filament, wherein the radiopaque filament provides improved external imaging of the filament in a body; and (ii) arranging the radiopaque filament for permanent attachment to a hollow tubular structure in a linear direction traverse to a longitudinal length of the tubular structure, the tubular structure providing a tubular wall defining an interior surface and an exterior surface and having opposed open first and second ends.

The method of this aspect of the invention desirably may include preparing the radiopaque filament by compounding a radiopaque powder with a polymeric material. Desirably, the radiopaque powder includes a radiopaque material selected from gold, barium sulfate, ferritic particles, platinum, platinum-tungsten, palladium, platinum-iridium, rhodium, tantalum or combinations thereof.

The method of this aspect of the invention desirably may include terminating the filament at the second end, arranging the filament at the first end in a series of closed loops with each loop having an apex defining a bend in one of the filaments and having an opposed base defined by crossing of adjacent filaments, and offsetting longitudinally the apex of adjacent closed loops from one and the other.

The method of this aspect of the invention desirably also may include arranging a plurality of polymeric radiopaque filaments in a helix configuration about a centerline of the tubular structure with a common axis, the plurality of polymeric radiopaque filaments arranged in a same linear direction.

The method of this aspect of the invention desirably may include preparing the polymeric radiopaque filaments by compounding a radiopaque powder with a polymeric material prior to extruding the filament. Desirably, the radiopaque powder includes a radiopaque material selected from gold, barium sulfate, ferritic particles, platinum, platinum-tungsten, palladium, platinum-iridium, rhodium, tantalum or combinations thereof.

The method of this aspect of the present invention desirably may include partially or fully coating or covering the stent with a polymeric material. The covering may be in the form of a partial or full cover or liner, such as a tubular structure which may be a conduit for liquid and/or prevent tissue ingrowth from encroaching on the stent lumen. Desirably, the covered stent or stent-graft is a polymeric material. The polymeric material may be selected from polyester, polypropylene, polyethylene, polyurethane, polynaphthalene, polytetrafluoroethylene, expanded polytetrafluoroethylene, silicone, and combinations thereof.

The method desirably may include mixing a radiopaque powder in a silicone bath, such that, the coating includes radiopaque particles.

The stents and methods of the present invention may be used at strictures or damaged vessel sites. Such sites may suitably include bodily tissue, bodily organs, vascular lumens, non-vascular lumens and combinations thereof, such as, but not limited to, in the coronary or peripheral vasculature, esophagus, trachea, bronchi, colon, biliary tract, urinary tract, prostate, brain, stomach and the like.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a perspective view of a hollow, tubular stent according to the present invention.

FIG. 2 is an expanded view of a wall portion of the stent of FIG. 1 taken along the 2-2 axis showing a plurality of stent filaments.

FIG. 3 depicts a braided stent with a closed-end loop design having a plurality of welds at the closed end according to the present invention.

FIG. 4 depicts a thirty-six filament braided stent that includes radiopaque and non-radiopaque filaments.

FIGS. 5a-d illustrate a perpendicular view of the stent of FIG. 4 having four radiopaque filaments (2CW and 2CCW), three radiopaque filaments, four radiopaque filaments and six radiopaque filaments, respectively.

FIGS. 6a-d illustrate a rotated 15 degree view of the stent of FIG. 4 having four radiopaque filaments (2CW and 2CCW), three radiopaque filaments, four radiopaque filaments and six radiopaque filaments, respectively.

FIGS. 7a-d illustrate a rotated 30 degree view of the stent of FIG. 4 having four radiopaque filaments (2CW and 2CCW), three radiopaque filaments, four radiopaque filaments and six radiopaque filaments, respectively.

FIGS. 8a-d illustrate a rotated 45 degree view of the stent of FIG. 4 having four radiopaque filaments (2CW and 2CCW), three radiopaque filaments, four radiopaque filaments and six radiopaque filaments, respectively.

FIG. 9 depicts a stent having a covering of silicone according to the present invention.

FIG. 10 is a cross-sectional view of the stent of FIG. 8 showing an outer covering of silicone about the stent.

FIG. 11 is a cross-sectional view of the stent of FIG. 9 showing an inner covering of silicone about the stent.

Like reference symbols in the various drawings indicate like elements.

### DETAILED DESCRIPTION OF THE PREFERRED EMBODIMENTS

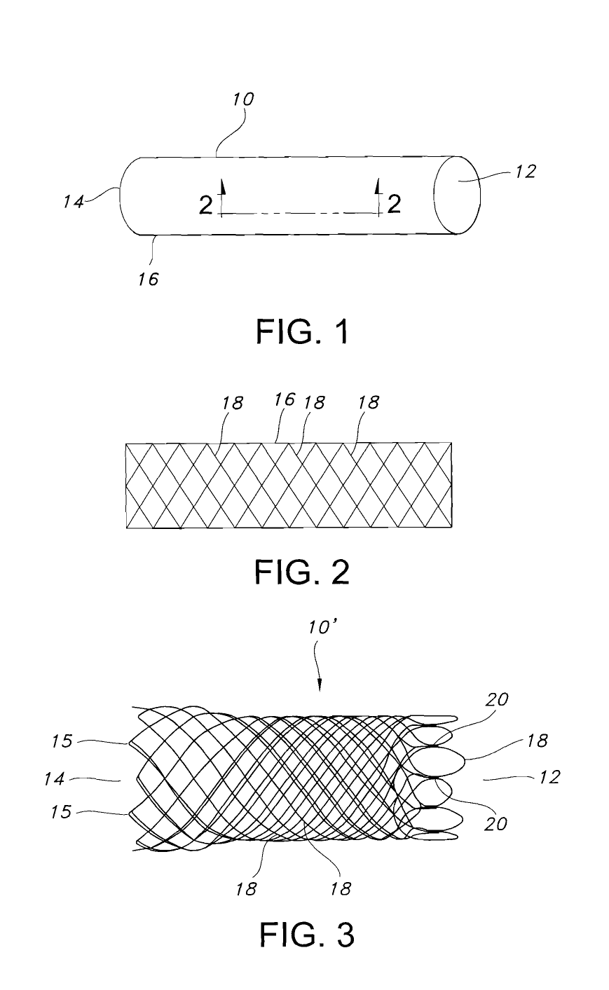

Referring now to FIG. 1, a stent 10 according to the present invention is disclosed. As shown in FIG. 1, the stent 10 includes a hollow tubular structure having opposed open ends 12, 14 and a tubular wall 16. A portion 2-2 of the tubular wall 16 is shown in FIG. 2 having a plurality of filaments or threads 18 which form the tubular wall 16. Tubular wall 16 is a distensible, open walled structure formed of filaments. The wall structure is radially expandable from a smaller radius to a larger radius. The radial expansion may occur as a result of the movement of filaments relative to one another or by plastic deformation of the filament material. The elongate filaments 18 traverse the length of the stent 10 in a direction traverse to the longitudinal length of the stent 10. The filaments 18 may be formed into the tubular wall 16 by braiding the filaments 18, winding the filaments 18, knitting the filaments 18, and combinations thereof. In some preferred embodiments, the filaments 18 are braided to form the tubular wall 16.

As used herein the term braiding and its variants refer to the diagonal intersection of elongate filaments, such as elongate wires, wire composites and polymeric filaments, so that each filament passes alternately over and under one or more of the other filaments, which is commonly referred to as an intersection repeat pattern. Useful braiding patterns include, but are not limited to, a diamond braid having a 1/1 intersection repeat pattern, a regular braid having a 2/2 intersection repeat pattern or a hercules braid having a 3/3 intersection repeat pattern. The passing of the filaments under and over one and the other results in slidable filament crossings that are not mechanically engaged or constrained.

Referring now to FIG. 3, in one preferred embodiment, the stent 10 is formed such that the elongate filaments 18 terminating at open end 12 may be mated and adjacently mated filaments may be secured to one and the other by welds 20 or by other suitable means. For example, in one preferred embodiment, the filaments 18 may be welded together through use of a welding material. In another preferred embodiment, the filaments 18 are heatingly and/or meltably fused together without the use of a welding material. In yet other preferred embodiments, for example, the filaments 18 are mechanically joined, such as, through the use of a small-sized or micro-fabricated clamp, crimpable tube, hypotube, and the like. Various techniques for welding filaments are known in the art.

The stent 10 shown in FIG. 3 is a braided stent that includes filaments 18 that are fully or partially composite filaments or wires 18. The filaments 18 provide improved external imaging of the stent in the body. Desirably, the enhanced visibility is enhanced radiopacity to provide improved fluoroscopic or x-ray visualization of the filaments in the body. Enhanced radiopacity may be achieved by using the below-described radiopaque materials in combination with a biocompatible and/or polymeric stent material. Such radiopaque materials are believed to be more visible under fluoroscopic or x-ray visualization due to their higher density than the corresponding biocompatible and/or polymeric stent material.

As shown in FIG. 3, in one preferred embodiment, the stent filaments 18 at the open end 14 may be bent to form closed loop ends 15 thereat. The loop ends 15 are substantially angular having approximately or about a 90° bend. The radius of curvature at the point of the bend is desirably minimized. In other words, the loop end 15 desirably has an angularly bent portion between substantially straight filament portions that do not otherwise have a portion with a significant radius of curvature. The loop ends 15, however, are not limited to angular bends of 90° and other bend angles may suitably be used. For example, angular bends with a bend angle from about 30° to about 150° are also useful. Other useful bend angles include from about 60° to about 120°, from about 70° to about 110°, from about 80° to about 100°, from about 85° to about 95°, and the like. The loop ends 15, however, are not limited to substantially angular bend-containing loops and other shaped loop ends, such as semi-circular, semi-elliptical and other smoothly curved or substantially smoothly curved loops, including but not limited to cathedral-shaped loops, may suitably be used.

The stent 10 depicted in FIG. 3 includes twenty-four filaments 18 of biocompatible material. In one preferred embodiment, the filaments 18 are relatively thin at a diameter of about 0.011 inches. The number of filaments and the diameters of the filaments, which may be the same or different, depicted in FIG. 3 are not limiting, and other numbers of filaments and other filament diameters may suitably be used. Desirably, an even number of filaments are used, for example from about 10 to about 36 wires.

The filaments 18 are made from a biocompatible material or biocompatible materials. Useful biocompatible materials include biocompatible metals, biocompatible alloys and biocompatible polymeric materials, including synthetic biocompatible polymeric materials and bioabsorbable or biodegradable polymeric materials. Desirably, the filaments 18 are biocompatible metals or alloys made from, but not limited to, nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber. Useful and nonlimiting examples of bioabsorbable or biodegradable polymeric materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like. In one preferred embodiment, for example, radiopaque materials such as barium sulfate and bismuth trioxide are compounded with the biocompatible material and are extruded into radiopaque filaments using a double extruder. Various radiopaque materials and their salts and derivatives may be used including, without limitation, bismuth, barium and its salts such as barium sulfate, tantalum, tungsten, gold, platinum and titanium, to name a few. Additional useful radiopaque materials may be found in U.S. Pat. No. 6,626,936, which is herein incorporated in its entirety by reference.

The filaments 18 made from polymeric materials also may include radiopaque materials, such as metallic-based powders or ceramic-based powders, particulates or pastes which may be incorporated into the polymeric material. The radiopaque material may be blended with the polymer composition from which the polymeric filament is formed, and subsequently fashioned into the stent. For example, in some preferred embodiments, a radiopaque powder is added to the polymeric material at extrusion time using a double screw extruder to form stent filaments. The radiopaque powder typically includes at least one element having a high atomic number such as bismuth, barium, tantalum, tungsten, gold, platinum.

For example, compounding approximately 50 to 70% weight of tantalum with polymeric material provides a filament comprising approximately 5 to 10% volume tantalum. Desirably, the low volume content of tantalum ensures that the filament maintains acceptable mechanical properties while being radiopaque.

In one preferred embodiment, the radiopaque filaments of the present invention include a longitudinal outer member concentrically disposed about a central core that extends along an axis of the outer member. Preferably, the outer member is formed of a metal, such as nitinol, that exhibits desirable properties, such as high elasticity and biocompatibility. The surface of the outer member may include a non-metal coating of, e.g., fluorocarbons, silicones, hydrophilic and lubricous biocompatible materials.) The central core of the radiopaque filaments includes a metal, such as tantalum, with a density greater than the longitudinal member to enhance the radiopacity of the filament and thus the stent from which it is formed. Preferably, the core is bonded to and substantially enclosed by the outer member such that the core does not have any substantial exposed surface and therefore does not contact body tissue when positioned within the body during use. In one preferred embodiment, the core is formed as a continuous solid member in intimate contact with and bonded to the interior portions of the outer member without the formation of substantial voids between the core and outer member. The core material preferably enhances the radiopacity of the filament but preferably does not substantially affect the mechanical performance of the filament.

In another preferred embodiment, the radiopaque filaments are formed as composite filaments including a central radiopaque core, an outer member, and an intermediate member between the core and the outer member. The intermediate member provides a barrier between the core and the outer member, and may be useful in composite filaments employing core and outer member materials that would be incompatible if contiguous, e.g. due to a tendency to form intermetallics.

In yet another preferred embodiment, the radiopaque filaments are formed as composite elements having a central radiopaque core, a structural outer member and a relatively thin annular outer cover layer. Suitable materials for the cover layer include tantalum, platinum, iridium, niobium, titanium and stainless steel.

The radiopaque polymeric stent of the present invention may be formed in various designs. For example, in one preferred embodiment, the stent is a flexible self-expandable stent that includes inside and outside stent walls each fabricated by knitting memory alloy filaments into a net-like structure with a first filament zigzagged and a second filament zigzagged at a plurality of interlocked points with intersecting points there between. Advantageously, the configuration of the first and second filaments allows the stent walls to apply force against longitudinal contraction of the stent walls. Preferably, the interlocked points and the intersecting points form a plurality of diamond-shaped lattices in the structure of each stent wall. Preferably, the lattices are covered with radiopaque material. In one preferred embodiment, a tubing is fitted between the inside and outside stent walls, with each of the overlapped ends of the tubing and the stent walls being integrating into a single structure.

In another preferred embodiment, the radiopaque polymeric stent is formed from a single wire. The stent may be formed by either hand or machine weaving. The stent may be created by bending shape memory filaments around tabs projecting from a template, and weaving the ends of the filaments to create the body of the stent such that the filaments cross each other to form a plurality of angles. Preferably, at least one of the angles is formed obtuse. The value of the obtuse angle may be increased by axially compressing the stent structure.

In another preferred embodiment, the radiopaque polymeric stent of the present invention includes a first tubular structure having a first inner diameter and a central axis, a second tubular structure connected to one end of the first tubular structure and having a second inner diameter, and a valve assembly that may prevent undesirable matter from entering the stent. The valve assembly preferably includes first, second and third valve members that are extended from the central axis to an inner circumference wall of the first tubular structure and are spaced away from each other at an angle of approximately 120 degrees in a circumference direction of the first tubular structure. In one preferred embodiment, the first, second and third valve members are provided with first, second and third passages, respectively, and a supporting valve member for connecting lower ends of the first, second and third valve members to an inner circumference wall of the first tubular structure.

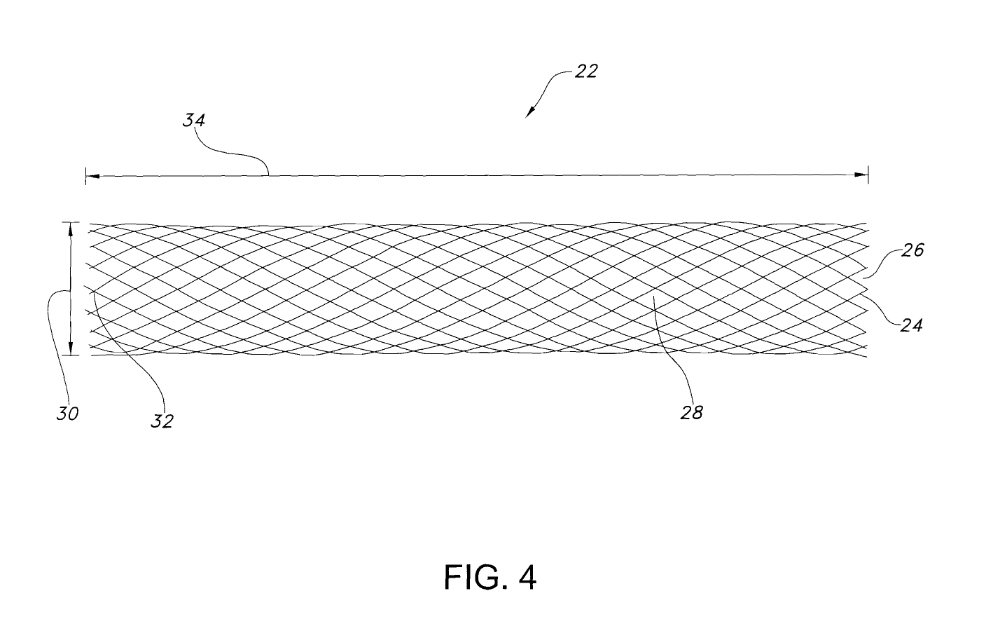

Referring now to FIG. 4, an example 36-filament braided stent 22 having both radiopaque and non-radiopaque filaments is shown. The filaments are braided in a helix pattern of 18-filaments braided clock-wise (CW) 24 and 18-filaments braided counter-clockwise (CCW) 26. In one preferred embodiment, the filaments 24, 26 are about equally spaced 28 from one another. The helix configuration includes a diameter 30 of about 15 mm. At this diameter, the pitch of the stent is approximately 85 mm and the radial spacing 32 at the crossing of filaments 24, 26 is approximately 20° degrees. The length 34 of the stent 22 is about 85 mm.

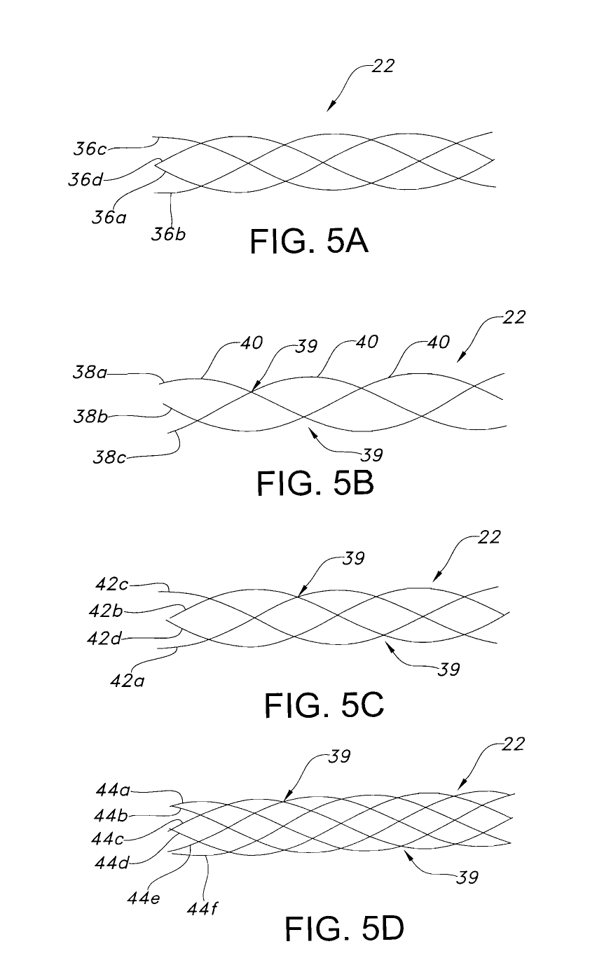

FIGS. 5a-d depict a perpendicular view of various arrangements of radiopaque filaments included in the stent 22 viewed under fluoroscope equipment. For example, FIG. 5a illustrates a perpendicular view of four radiopaque filaments 36a, 36b, 36c, 36d attached to the stent. As shown in FIG. 5a, two radiopaque filaments 36a, 36b are arranged in a first linear direction 2CW (e.g., clock-wise) and the two radiopaque filaments 36c, 36d are arranged in a second linear direction 2CCW (e.g., counter clockwise) opposite the first linear direction. The four filaments 36a, 36b, 36c and 36d are spaced at approximately 90° degrees apart at their furthest points and cross at two points 180° degrees apart.

FIG. 5b depicts a perpendicular view of three radiopaque filaments 38a, 38b, 38c that are approximately equally spaced from one another and are arranged in a first linear direction. In this embodiment, the radiopaque filaments 38a, 38b, 38c are braided into the stent 22 at about 120° degrees apart. As shown in FIG. 5b, a void area 39 exists between the peaks 40 of the three radiopaque filaments 38a, 38b, and 38c. The void area 39 represents approximately twenty-five percent of the view.

FIG. 5c depicts a perpendicular view of four radiopaque filaments 42a, 42b, 42c and 42d that are all arranged in a first linear direction. In this embodiment, one radiopaque filament 42a is attached to the stent at about a 0° degree position. The third radiopaque filament 42c is attached to the stent at about a 180° degree position. In one preferred embodiment, the second and fourth radiopaque filaments 42b, 42d are attached to the stent 22 at about 120° degrees apart. In another preferred embodiment, the second and fourth radiopaque filaments 42b, 42d are attached to the stent 22 at about 100° and 280° degrees apart, respectively.

FIG. 5d depicts a perpendicular view of six radiopaque filaments 44a, 44b, 44c, 44d, 44e and 44f that are all arranged in a first linear direction and are attached to the stent 22 at approximately 60° degrees apart. As shown in FIGS. 5a and 5c, the radiopaque image of each pattern's radiopaque filaments appears similar and each stent's void area 39 is reduced to about 15% percent of the stent image. The radiopaque stent of FIG. 5d has only about a five percent void area 39.

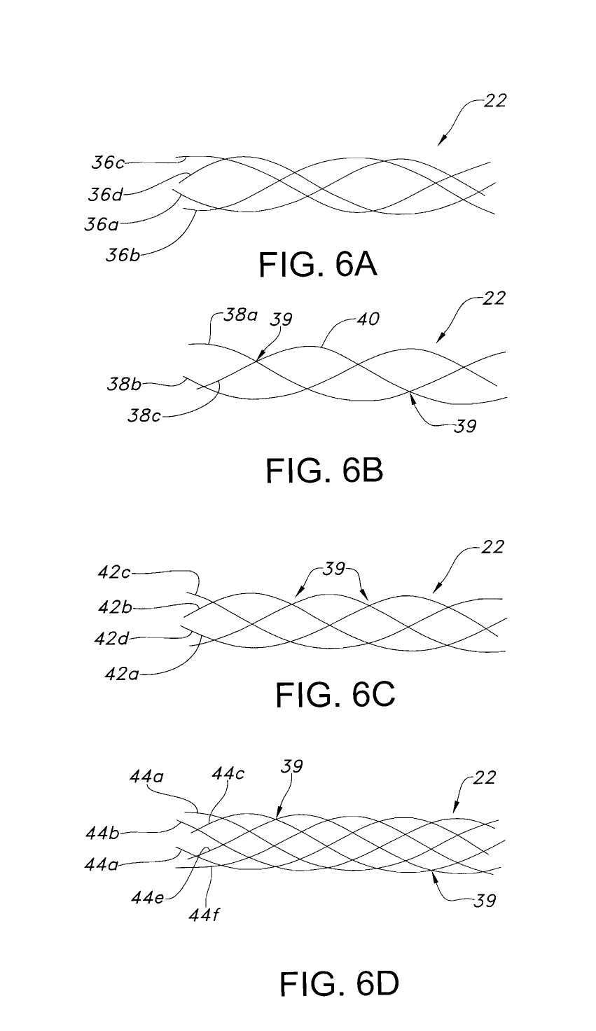

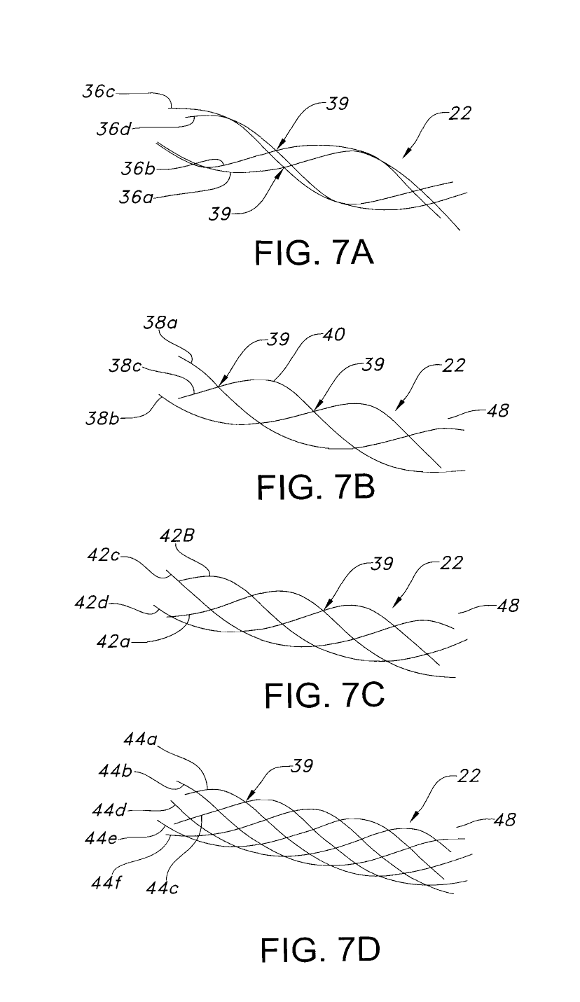

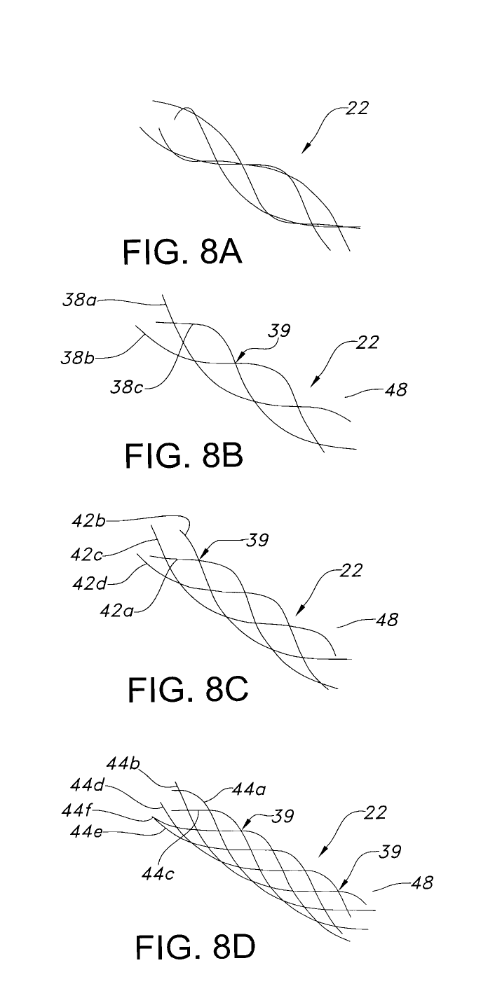

FIGS. 6a-d show the patterns of the radiopaque filaments of FIGS. 5a-d rotated at 15° degrees about two axes (Y-axis and Z-axis). FIGS. 7a-d and FIGS. 8a-d show the patterns of the radiopaque filaments of FIGS. 5a-d rotated at 30-degrees and 45-degrees about the same two axes, respectively.

As shown in FIG. 6a, the pattern image of radiopaque filaments of FIG. 5a distorts when the stent is viewed at a 15° degree angle. The image distortion in FIGS. 6b-d for the patterns shown in FIGS. 5b-d, respectively, when viewed at a 15° degree angle is minimal.

Referring now to FIG. 7a, when viewed at a 30° degree angle, the void area 39 of the radiopaque filament pattern of FIG. 5a increases to about 36% percent. The radiopaque patterns of FIGS. 5b-d, when viewed at a 30° degree angle and depicted in FIGS. 7b-d, respectively, appear skewed with additional void areas 39 on one side 48 of the stent. As shown in FIGS. 7b-d, the amount of image distortion depends on the direction of the filament and the position from where the stent is viewed.

FIGS. 8a-d show the patterns of the radiopaque filaments of FIGS. 5a-d rotated at a 45° degree angle, respectively. As shown in FIGS. 8b-d, the void area 39 of the radiopaque filament patterns remain skewed with additional void areas 39 on one side 48 of the stent. Desirably, radiopaque filaments are arranged in the stent in the same direction (e.g., linear direction) to minimize distortion of the pattern when viewing the stent from angled perspectives.

Although FIGS. 5a-8d depict various three, four, and six radiopaque filament patterns, the present invention is not limited to these embodiments. For example, in one preferred embodiment, a symmetrical pattern of 9-radiopaque filaments is arranged in a same linear direction in the stent resulting in about 99 percent of the stent being viewable from angled perspectives.

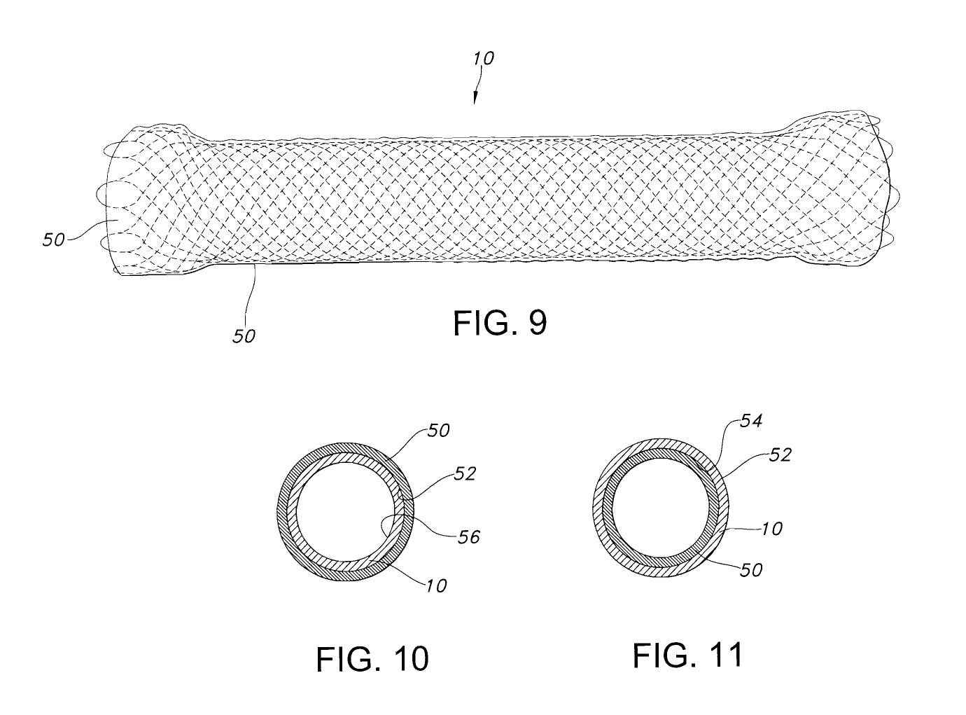

Referring now to FIG. 9, the stent 10 may be fully, substantially or partially covered or lined with a radiopaque polymeric material 50. The covering may be in the form of a tubular structure. Nonlimiting examples of polymeric coverings include silicone, polyurethane, polyethylene, polytetrafluoroetylene (PTFE) and expanded PTFE (ePTFE) and combinations and copolymers thereof. One nonlimiting example of a polymeric material is silicone. For example, in one preferred embodiment, the stent is covered with a silicon covering solution including radiopaque powder. In this preferred embodiment, radiopaque particles included in the powder are incorporated into the silicone covering providing improved radiopacity.

In another preferred embodiment, radiopaque material is added to the silicon covering solution by metallurgically alloying or by making clad composite structures. Radiopaque materials also may be filled into hollow cores, cavities or pores in the polymer matrix. Organic radiopaque powders containing elements or salts or oxides of elements such as bromine, iodine, iodide, barium, and bismuth also may be used instead of metal powders.

The radiopaque polymeric material 50 may be disposed on external surfaces 52 of the stent 10, as depicted in FIG. 10, or disposed on the internal surfaces 54 of the stent 10, as depicted in FIG. 11, or combinations thereof. The silicone covering may be suitably formed by dip coating the stent. The present invention is not limited to forming the silicone film by dip coating, and other techniques, such as spraying, may suitably be used. After applying the radiopaque silicone coating or film to the stent, the silicone may be cured. Desirably, the curing is low temperature curing, for example from about room temperature to about 90° C. for a short period of time, for example from about 10 minutes or more to about 16 hours. The cured radiopaque silicone covering may also be sterilized by electronic beam radiation, gamma radiation ethylene oxide treatment and the like. Further details of the curing and/or sterilization techniques may be found in U.S. Pat. No. 6,099,562, the content of which is incorporated herein by reference. Argon plasma treatment of the cured silicone may also be used.

With any embodiment of the stent 10, 22 of the present invention, the stent may be usable to maintain patency of a bodily vessel, such as in the coronary or peripheral vasculature, or non vascular lumens and ducts such as the esophagus, trachea, bronchi colon, small intestine, biliary tract, urinary tract, prostate, brain, and the like. Also, the stent 10, 22 may be treated with any of the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promotors (such as growth factor inhibitors, growth factor receptor antagonists, transcriptional activators, and translational promotors); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

In one aspect of the present invention, an implantable stent is provided. The stent includes at least one radiopaque filament arranged for permanent attachment to a hollow tubular structure in a linear direction traverse to a longitudinal length of the hollow tubular structure, the tubular structure having a tubular wall that defines an inner surface and an outer surface and opposing first open end and second open end, the at least one radiopaque filament comprising a radiopaque material and a polymeric material. Preferably, the at least one radiopaque filament improves external imaging of the tubular structure on fluoroscope or x-ray imaging equipment.

Desirably, the implantable radiopaque stent includes a plurality of radiopaque filaments.

The plurality of radiopaque filaments may be arranged in a helix configuration about a centerline of the tubular structure with a common axis. Preferably, the plurality of radiopaque filaments form the tubular structure.

The stent of this aspect of the present invention desirably may have a braided hollow tubular structure. Preferably, the stent of the present invention desirably is biodegradable.

The stent of this aspect of the present invention desirably may also have the filaments terminate at the second end, wherein the filaments at the first end are arranged in a series of closed loops with each loop having an apex defined by a bend in one of the filaments and having an opposed base defined by crossing of adjacent filaments, and further wherein the apex of adjacent closed loops are longitudinally offset from one and the other.

The stent of this aspect of the present invention desirably may have the radiopaque material selected from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof. Desirably, the radiopaque material is a radiopaque powder.

The stent of this aspect of the present invention desirably may have the polymeric material selected from the group consisting of polyester, polypropylene, polyethylene, polyurethane, polynaphthalene, polytetrafluoroethylene, expanded polytetrafluoroethylene, silicone, and combinations thereof.

The stent of this aspect of the present invention desirably may have the at least one radiopaque filament include a radiopaque material and a bioabsorbable material. Desirably, the bioabsorbable material is adapted to degrade in vivo. The bioabsorbable material may be selected from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

Desirably, the radiopaque material is selected from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof.

The stent of this aspect of the present invention desirably may have the tubular structure covered with a polymeric material. Desirably, the polymeric material is selected from the group consisting of polyester, polypropylene, polyethylene, polyurethane, polynaphthalene, polytetrafluoroethylene, expanded polytetrafluoroethylene, silicone, and combinations thereof.

The stent of this aspect of the present invention desirably may have the polymeric material including radiopaque particles.

The stent of this aspect of the present invention desirably may further include a polymeric covering. Desirably, the polymeric covering is biodegradable.

The stent of this aspect of the present invention desirably may further have all of the at least one radiopaque filaments arranged in a first linear direction.

In another aspect of the present invention, an implantable stent is provided that includes a plurality of elongate radiopaque filaments braided to form a hollow tubular structure having a tubular wall that defines an inner surface and an outer surface and opposing first open end and second open end. Desirably, the stent also includes a polymeric covering over the tubular structure.

The stent of this aspect of the present invention preferably includes radiopaque material in the polymeric covering. Desirably, the polymeric covering is prepared by mixing a radiopaque powder with a polymeric material.

The stent of this aspect of the present invention preferably includes at least one of the plurality of radiopaque filaments having a radiopaque material and a biocompatible material. Desirably, the biocompatible material is selected from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof. Desirably, the radiopaque material may be selected from the group consisting of gold, barium sulfate, ferritic particles, platinum, platinum-tungsten, palladium, platinum-iridium, rhodium, tantalum and combinations thereof.

The stent of this aspect of the present invention preferably includes the at least one of the plurality of radiopaque filaments having a radiopaque material and a polymeric material. Desirably, the radiopaque material is selected from the group consisting of gold, barium sulfate, ferritic particles, platinum, platinum-tungsten, palladium, platinum-iridium, rhodium, tantalum and combinations thereof. Preferably, the radiopaque material is a radiopaque powder.

The stent of this aspect of the present invention preferably includes selecting the polymeric material from the group consisting of polyester, polypropylene, polyethylene, polyurethane, polynaphthalene, polytetrafluoroethylene, expanded polytetrafluoroethylene, silicone, and combinations thereof.

The stent of this aspect of the present invention preferably may include at least one of the plurality of radiopaque filaments having a polymer or copolymer.

In yet another aspect of the present invention, a method for making an implantable stent includes providing at least one radiopaque filament, and arranging the at least one radiopaque filament for permanent attachment to a hollow tubular structure in a linear direction traverse to a longitudinal length of the tubular structure. Preferably, the tubular structure provides a tubular wall defining an interior surface and an exterior surface and having opposed open first and second ends.

The method of this aspect of the invention may further include providing a plurality of radiopaque filaments. Desirably, the method may also include arranging a plurality of radiopaque filament in a helix configuration about a centerline of the tubular structure with a common axis.

The method of this aspect of the present invention may include braiding a plurality of radiopaque filaments to form the tubular structure. Preferably, forming the at least one radiopaque filament comprises from a radiopaque material and a polymeric material.

The method of this aspect of the present invention may include selecting the polymeric material from the group consisting of polyester, polypropylene, polyethylene, polyurethane, polynaphthalene, polytetrafluoroethylene, expanded polytetrafluoroethylene, silicone, and combinations thereof. Desirably, the method may also include compounding the radiopaque material with the polymeric material. The radiopaque material may be a radiopaque powder.

The method of this aspect of the present invention may include selecting the radiopaque material from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof.

The method of this aspect of the present invention may further include forming the at least one radiopaque filament comprises from a radiopaque material and a biocompatible material. Desirably, the method also includes adapting the biocompatible material to degrade in vivo.

The method of this aspect of the present invention may include selecting the biocompatible material from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

Desirably, the method of this aspect of the invention includes forming the at least one radiopaque filament from a polymer or copolymer.

The method of this aspect of the present invention may include forming a cover for the tubular structure by covering the tubular structure with a polymeric material. The method of this aspect of the invention may also include mixing a radiopaque powder in a silicon solution, such that, the cover includes radiopaque particles.

The method of this aspect of the present invention may include terminating the filament at the second end, arranging the filament at the first end in a series of closed loops with each loop having an apex defining a bend in one of the filaments and having an opposed base defined by crossing of adjacent filaments, and offsetting longitudinally the apex of adjacent closed loops from one and the other.

In yet another aspect of the present invention, a method for making an implantable stent includes braiding a plurality of elongate filaments to form a hollow tubular structure having a tubular wall that defines an inner surface and an outer surface and opposing first open end and second open end, and covering the tubular structure with a polymeric material including radiopaque particles, wherein the radiopaque particles improve external imaging of the tubular structure on fluoroscope or x-ray imaging equipment.

The method of this aspect of the present invention may include mixing a radiopaque powder with the polymeric material for covering the tubular structure. The method of this aspect of the present invention may also include forming the filaments by compounding a radiopaque material with a polymer material and/or biocompatible material.

Further, with any embodiment of the stent 10, 22, the general tubular shape may be varied. For example, the tubular shape may have a varied diameter, an inwardly flared end, an outwardly flared end and the like. Further, the ends of the stent may have a larger diameter than the middle regions of the stent. A braided stent with outwardly flared ends is further described in U.S. Pat. No. 5,876,448, the contents of which are incorporated herein by reference. The invention being thus described, it will now be evident to those skilled in the art that the same may be varied in many ways. Such variations are not to be regarded as a departure from the spirit and scope of the invention and all such modifications are intended to be included within the scope of the following claims.

What is claimed is:

1. An implantable radiopaque stent comprising:
at least one radiopaque filament arranged for permanent attachment to a hollow tubular structure in a linear direction traverse to a longitudinal length of the hollow tubular structure, the tubular structure having a tubular wall that defines an inner surface and an outer surface and opposing first open end and second open end, wherein the at least one radiopaque filament improves external imaging of the tubular structure on fluoroscope or x-ray imaging equipment.

2. The implantable radiopaque stent of claim 1, comprising a plurality of radiopaque filaments.

3. The implantable radiopaque stent of claim 2, wherein the plurality of radiopaque filaments are arranged in a helix configuration about a centerline of the tubular structure with a common axis.

4. The implantable radiopaque stent of claim 2, wherein the plurality of radiopaque filaments form the tubular structure.

5. The implantable radiopaque stent of claim 1, wherein the hollow tubular structure is braided.

6. The implantable radiopaque stent of claim 2, wherein the filaments terminate at the second end, wherein the filaments at the first end are arranged in a series of closed loops with each loop having an apex defined by a bend in one of the filaments and having an opposed base defined by crossing of adjacent filaments, and further wherein the apex of adjacent closed loops are longitudinally offset from one and the other.

7. The implantable radiopaque stent of claim 1, wherein the at least one radiopaque filament comprises a radiopaque material and a polymeric material.

8. The implantable radiopaque stent of claim 7, wherein the radiopaque material is selected from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof.

9. The implantable radiopaque stent of claim 7, wherein the radiopaque material is a radiopaque powder.

10. The implantable radiopaque stent of claim 7, wherein the polymeric material is selected from the group consisting of polyester, polypropylene, polyethylene, polyurethane, polynaphthalene, polytetrafluoroethylene, expanded polytetrafluoroethylene, silicone, and combinations thereof.

11. The implantable radiopaque stent of claim 1, wherein the at least one radiopaque filament comprises a radiopaque material and a bioabsorbable material.

12. The implantable radiopaque stent of claim 11, wherein the bioabsorbable material is adapted to degrade in vivo.

13. The implantable radiopaque stent of claim 11, wherein the at least one radiopaque filament comprises a polymer or copolymer.

14. The implantable radiopaque stent of claim 11, wherein the bioabsorbable material is selected from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

15. The implantable radiopaque stent of claim 11, wherein the radiopaque material is selected from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof.

16. The implantable radiopaque stent of claim 1, wherein the tubular structure is covered with a polymeric material.

17. The implantable radiopaque stent of claim 16, wherein the polymeric material is selected from the group consisting of polyester, polypropylene, polyethylene, polyurethane, polynaphthalene, polytetrafluoroethylene, expanded polytetrafluoroethylene, silicone, and combinations thereof.

18. The implantable radiopaque stent of claim 17, wherein the polymeric material includes radiopaque particles.

19. The implantable radiopaque stent of claim 1, further comprising a polymeric covering over the tubular structure.

20. The implantable radiopaque stent of claim 19, wherein the polymeric covering is biodegradable.

21. An implantable radiopaque stent comprising:
a plurality of elongate radiopaque filaments braided to form a hollow tubular structure having a tubular wall that defines an inner surface and an outer surface and opposing first open end and second open end; and
a polymeric covering over the tubular structure.

22. The implantable radiopaque stent of claim 21, wherein the polymeric covering includes radiopaque material.

23. The implantable radiopaque stent of claim 21, wherein the polymeric covering is prepared by mixing a radiopaque powder with a polymeric material.

24. The implantable radiopaque stent of claim 21, wherein at least one of the plurality of radiopaque filaments comprises a radiopaque material and a biocompatible material.

25. The implantable radiopaque stent of claim 24, wherein the biocompatible material is selected from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

26. The implantable radiopaque stent of claim 24, wherein the radiopaque material is selected from the group consisting of gold, barium sulfate, ferritic particles, platinum, platinum-tungsten, palladium, platinum-iridium, rhodium, tantalum and combinations thereof.

27. The implantable radiopaque stent of claim 21, wherein the at least one of the plurality of radiopaque filaments comprises a radiopaque material and a polymeric material.

28. The implantable radiopaque stent of claim 27 wherein the radiopaque material is selected from the group consisting of gold, barium sulfate, ferritic particles, platinum, platinum-tungsten, palladium, platinum-iridium, rhodium, tantalum and combinations thereof.

29. The implantable radiopaque stent of claim 27 wherein the radiopaque material is a radiopaque powder.

30. The implantable radiopaque stent of claim 27, wherein the polymeric material is selected from the group consisting of polyester, polypropylene, polyethylene, polyurethane, polynaphthalene, polytetrafluoroethylene, expanded polytetrafluoroethylene, silicone, and combinations thereof.

31. The implantable radiopaque stent of claim 21, wherein at least one of the plurality of radiopaque filaments comprises a polymer or copolymer.

32. A method for making an implantable stent comprising:
providing at least one radiopaque filament; and
arranging the at least one radiopaque filament for permanent attachment to a hollow tubular structure in a linear direction traverse to a longitudinal length of the tubular structure, the tubular structure providing a tubular wall defining an interior surface and an exterior surface and having opposed open first and second ends.

33. The method of claim 32, comprising providing a plurality of radiopaque filaments.

34. The method of claim 32, comprising arranging a plurality of radiopaque filament in a helix configuration about a centerline of the tubular structure with a common axis.

35. The method of claim 32, comprising braiding a plurality of radiopaque filaments to form the tubular structure.

36. The method of claim 32, comprising forming the at least one radiopaque filament comprises from a radiopaque material and a polymeric material.

37. The method of claim 36, comprising selecting the polymeric material from the group consisting of polyester, polypropylene, polyethylene, polyurethane, polynaphthalene, polytetrafluoroethylene, expanded polytetrafluoroethylene, silicone, and combinations thereof.

38. The method of claim 36, comprising compounding the radiopaque material with the polymeric material.

39. The method of claim 36, wherein the radiopaque material is a radiopaque powder.

40. The method of claim 36, comprising selecting the radiopaque material from the group consisting of gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof.

41. The method of claim 32, comprising forming the at least one radiopaque filament comprises from a radiopaque material and a biocompatible material.

42. The method of claim 41, comprising adapting the biocompatible material to degrade in vivo.

43. The method of claim 42, comprising selecting the biocompatible material from the group consisting of poly-L-lactide, poly-D-lactide, polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly (alpha-hydroxy acid) and combinations thereof.

44. The method of claim 41, comprising forming the at least one radiopaque filament from a polymer or copolymer.

45. The method of claim 32, comprising forming a cover for the tubular structure by covering the tubular structure with a polymeric material.

46. The method of claim 44, comprising mixing a radiopaque powder in a silicon bath, such that, the cover includes radiopaque particles.

47. The method of claim 32, comprising:
terminating the filament at the second end;
arranging the filament at the first end in a series of closed loops with each loop having an apex defining a bend in one of the filaments and having an opposed base defined by crossing of adjacent filaments; and
offsetting longitudinally the apex of adjacent closed loops from one and the other.

48. A method for making an implantable stent comprising:
braiding a plurality of elongate filaments to form a hollow tubular structure having a tubular wall that defines an inner surface and an outer surface and opposing first open end and second open end; and
covering the tubular structure with a polymeric material including radiopaque particles, wherein the radiopaque particles improve external imaging of the tubular structure on fluoroscope or x-ray imaging equipment.

49. The method of claim 48, wherein covering the tubular structure comprises mixing a radiopaque powder with the polymeric material.

50. The method of claim 48, comprising forming the filaments by compounding a radiopaque material with a polymer material.

51. The method of claim 48, comprising forming the filaments by compounding a radiopaque material with at least one of a polymer and biocompatible material.
